(19) United States
(12) Patent Application Publication
(10) Pub. No.: US 2011/0213021 A1
(43) Pub. Date: Sep. 1, 2011

(54) COMPOSITIONS AND METHODS FOR TREATING NOS-ASSOCIATED DISEASES

(75) Inventors: Mahesh Kandula, Andhra Pradesh (IN); Mary E. Vaman Rao, Hopkinton, MA (US)

(73) Assignee: Indigene Pharmaceuticals, Inc., Westborough, MA (US)

(21) Appl. No.: 12/920,999

(22) PCT Filed: Mar. 3, 2009
(86) PCT No.: PCT/US09/01401

§ 371 Date: May 2, 2011

Related U.S. Application Data
(60) Provisional application No. 61/068,166, filed on Mar. 4, 2008.

(51) Int. Cl. A61K 31/385; C07D 339/04; A61P 25/28; A61P 25/16; A61P 25/14; A61P 3/10

(52) U.S. Cl. 514/440; 549/39

(57) ABSTRACT

The disclosures herein provide lipoic acid salts, as well as polymorphs, solvates, and hydrates thereof. These salts may be formulated as pharmaceutical compositions. The pharmaceutical compositions may be formulated for oral administration, transdermal administration, or injection. Such compositions may be used to treat NOS-associated diseases such as inflammatory diseases, metabolic diseases and neurodegenerative diseases.

### RELATED APPLICATIONS

This application claims the benefit of U.S. Provisional Application No. 61/068,166, filed Mar. 4, 2008, the specification of which is hereby incorporated herein by reference in its entirety.

### BACKGROUND

Misregulation of the ubiquitous enzyme nitric oxide synthase (NOS) is implicated in numerous diseases. In particular, inducible nitric oxide synthase (iNOS) is frequently misregulated. Neurodegenerative diseases, inflammatory diseases, and metabolic diseases such as diabetes are often characterized by elevated NOS activity. Such diseases may be treated with compositions that lower NOS activity. However, currently available NOS-inhibiting therapies often have undesirable side effects.

Nitric oxide (NO) is produced by NOS, and NO participates in inflammatory and autoimmune mediated tissue destruction, such as that observed in rheumatoid arthritis. Inflammatory processes in vivo inter-regulate the expression and function of NOS. NO formation is increased during inflammation (in diseases such as arthritis, ulcerative colitis, and Crohn's disease), and several classic inflammatory symptoms (erythema, vascular leakiness) are reversed by NOS inhibitors.

Elevated NOS levels, including iNOS levels, are also typical of diabetes. In fact, abnormally elevated glucose levels in diabetic patients can damage cells through the generation of NO, as well as other nitrosative species, reactive oxygen species, H₂O₂, and ketoaldehydes. These compounds have been associated with the development of diabetic complications related to the production and accumulation of advanced glycation end-products (AGE) by Maillard reaction in tissues. AGEs deposited in blood vessels produce free radicals, and can degrade vessel lipids and accelerate atherogenesis in hyperglycemic diabetic patients. Furthermore, AGEs in diabetic patients play an important role in the development of diabetic complications such as nephropathy, neuropathy, retinopathy, and diabetic foot ulcers. While not wishing to be bound by theory, NOS and AGEs may act together to cause tissue-damage in diabetic patients.

In the nervous system, high concentrations of NO (generated by NOS) are potently neurotoxic. This is of particular significance in neurodegenerative conditions such as Alzheimer's disease, Parkinson's disease, Huntington's disease and Krabbe's disease. In addition, the overproduction of NO by NOS has been tightly linked to neuroinflammation and neurodegeneration associated with traumatic injuries and production of proinflammatory cytokines.

There is currently a need in the art for new compositions to treat NOS-associated diseases. There is also a need for compositions designed to treat diseases associated with AGEs.

### SUMMARY OF THE INVENTION

The disclosures herein provide, inter alia, lipoic acid salts of the compounds of Formula I, II, III, or IV, as well as polymorphs, solvates, and hydrates thereof. These salts may be formulated as pharmaceutical compositions. The salts and pharmaceutical compositions may be formulated for oral administration, transdermal administration, or injection. Such compositions may be used to treat NOS-associated diseases such as, for example, inflammatory diseases, metabolic diseases, and neurodegenerative diseases.

The present application also provides a lipoic acid salt of a compound of Formula I:

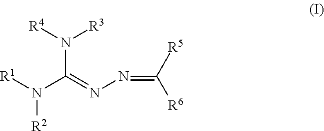

having a lipoate ion enriched for the R-(+) enantiomer, and wherein
R¹ and R², each independently, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, and a macromolecule, or R¹ and R², taken together, form ═C═O, ═CH—CHO, or ═C(R⁷)(R⁸);
R³ and R⁴, each independently, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, and a macromolecule, or R³ and R⁴, taken together, form ═C═O, ═CH—CHO, or ═C(R⁷)(R⁸);
R⁵ and R⁶, each independently, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, thioether, and a macromolecule;
R⁷ and R⁸, each independently, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, thioether, and a macromolecule.
   In certain embodiments, R¹ is H, R² is H, R³ is H, R⁴ is H, and
R⁵ and R⁶, each independently, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, thioether, thioketone, and a macromolecule.
   In some embodiments, R¹ is H, R² is H, R³ is H, R⁴ is H, R⁵ is CH₃ and R⁶ is CH₃.

In certain embodiments, the lower alkyl is a C₁-Cₒ-alkyl. In certain embodiments, the acyl is a formyl. In certain embodiments, the macromolecule is a polypeptide or oligopeptide. In certain embodiments, the polypeptide is an antibody. In certain embodiments, the salt is in crystalline form. In certain embodiments, the salt is substantially free of the S enantiomer of lipoate.

In addition, the present application describes the lipoic acid salt a compound of Formula II.

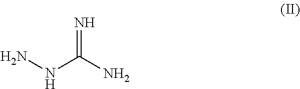

having a lipoate ion enriched for the R-(+) enantiomer.

The disclosures herein also relate to the lipoic acid salt of the compound of Formula III:

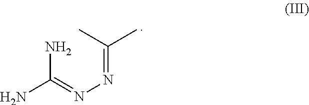

having a lipoate ion enriched for the R-(+) enantiomer.

In certain embodiments, the present disclosure provides the lipoic acid salt of a compound of Formula IV:

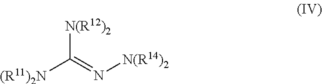

having a lipoate ion enriched for the R-(+) enantiomer, wherein
R¹¹, independently for each occurrence, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, and a macromolecule, or both occurrences of R¹¹ taken together form ═C═O, ═CH—CHO, or ═C(R¹⁵)₂;
R¹², independently for each occurrence, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, and a macromolecule, or both occurrences of R¹² taken together form ═C═O, ═CH—CHO, or ═C(R¹⁵)₂;
R¹⁴, independently for each occurrence, is selected from: H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, and a macromolecule, or both occurrences of R¹⁴ taken together form ═C═O, ═CH—CHO, or ═C(R¹⁵)_{2;}
R¹⁵, independently for each occurrence, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, and a macromolecule.

In one embodiment, at least one of R¹¹, R¹², and R¹⁴ is other than H.

In certain embodiments, R¹¹ is H, R¹² is H, and
R¹⁴, independently for each occurrence, is selected from: acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, and a macromolecule, or both occurrences of R¹⁴ taken together form ═C═O, ═CH—CHO, or ═C(R¹⁵)₂; and
R¹⁵, independently for each occurrence, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, and a macromolecule.

In certain embodiments, both occurrences of R¹⁴ are methyl.

In certain embodiments, both instances of R¹¹, taken together, are the moiety resulting from the formation of a Schiff base by reaction with a carbonyl, such as aldehydes, ketones, acetone, acetaldehyde, and in general any molecule having a carbonyl moiety. In certain embodiments, both instances of R¹², taken together, are the moiety resulting from the formation of a Schiff base by reaction with a carbonyl, such as aldehydes, ketones, acetone, acetaldehyde, and in general any molecule having a carbonyl moiety. In certain embodiments, both instances of R¹⁴, taken together, are the moiety resulting from the formation of a Schiff base by reaction with a carbonyl, such as aldehydes, ketones, acetone, acetaldehyde, and in general any molecule having a carbonyl moiety.

In certain embodiments, the lower alkyl is a C₁-C₆-alkyl. In certain embodiments, the acyl is a formyl. In certain embodiments, the macromolecule is a polypeptide or oligopeptide. In certain embodiments, the polypeptide is an antibody. In certain embodiments, the salt is in crystalline form. In certain embodiments, the salt is substantially free of the S enantiomer of lipoate.

This application also discloses a pharmaceutical composition comprising a pharmaceutically acceptable carrier and the lipoic acid salt of a compound of Formula I, II, III, or IV. The pharmaceutical composition may be formulated for systemic or topical administration. The pharmaceutical composition may be formulated for oral administration, injection, subdermal administration, or transdermal administration. The pharmaceutical composition may further comprise at least one of a pharmaceutically acceptable stabilizer, diluent, surfactant, filler, binder, and lubricant. The pharmaceutical composition may also include L-arginine.

In addition, the present application discloses a method of treating an NOS-associated disease comprising, administering to a patient in need thereof a therapeutically effective amount of a lipoic acid salt of a compound of Formula I, II, III, or IV. In addition, the instant application provides a method of reducing NOS activity comprising, administering to a patient in need thereof an effective amount of the lipoic acid salt of a compound of Formula I, II, III, or IV.

In certain embodiments, the disease is arthritis, Alzheimer's disease, Huntington's disease, or Parkinson's disease, diabetes, or a diabetic complication such as nephropathy, retinopathy, vasculopathy, neuropathy and diabetic foot ulcers. The disease may be, for instance, an inflammatory disease, a neurodegenerative disease, or a metabolic disease.

In certain embodiments, the lipoic acid salt is administered systemically, for example as a pill, capsule, injection, or patch.

Herein Applicants also disclose a kit comprising a pharmaceutical preparation that includes the lipoic acid salt of the compound of Formula I, II, III, or IV. In an alternative embodiment, the kit contains a first pharmaceutical composition comprising lipoic acid or a pharmaceutically acceptable salt thereof, and a second pharmaceutical composition comprising a compound of Formula I, II, III, or IV, or a pharmaceutically acceptable salt thereof.

The present application also discloses the use of a lipoic acid salt of the compound of Formula I, II, III, or IV, in the preparation of a medicament intended to treat NOS-associated diseases.

The present disclosure also provides a pharmaceutical composition, intended for systemic administration, comprising a pharmaceutically acceptable carrier and the lipoic acid salt of the compound of Formula I, II, III, or IV.

The present disclosure also contemplates prodrugs of the compositions disclosed herein, as well as pharmaceutically acceptable salts of said prodrugs.

In certain embodiments, the macromolecule is heparin. The macromolecule may also be a polypeptide or oligopeptide. This polypeptide may be an antibody. The antibody may be a therapeutic antibody or an antibody that targets the composition to a desired organ or tissue. In certain embodiments, the protein is albumin. The protein may be any therapeutic protein known in the art. The protein may incorporate non-natural amino acids. In certain embodiments, the macromolecule is a nucleic acid. This nucleic acid may be DNA, RNA, or a variant thereof such as a PNA or morpholino. The nucleic acid may be single-stranded or double-stranded. The nucleic acid may be an siRNA. The nucleic acid may be single stranded or double stranded. In yet other embodiments, the macromolecule is a polysaccharide. The macromolecule may, for example, improve the stability or bioavailability of the composition. The macromolecule may also be an additional therapeutic. This additional therapeutic may be intended to treat the disease for which the lipoic acid salt is being administered, boost one or more activities of the lipoic acid salt, or reduce one or more activities of the lipoic acid salt.

In addition, the present disclosure provides compositions comprising aminoguanidine and lipoic acid (or lipoate), wherein the aminoguanidine and lipoic acid (or lipoate) do not form a salt. In addition, the present disclosure provides compositions comprising aminoguanidinium and lipoic acid (or lipoate), wherein the aminoguanidinium and lipoic acid (or lipoate) do not form a salt.

Additionally, herein Applicants disclose certain methods of producing lipoic acid salts of the compounds of Formula I, II, III, or IV. In certain aspects, lipoic acid is combined with a salt of the compound of Formula I, II, III, or IV. In certain embodiments, the salt of the compound of Formula I, II, III, or IV is a hydrochloride salt.

In certain embodiments, the present disclosure provides methods of treatment with other preparations of lipoic acid salts. In certain embodiments, the therapeutic preparation may be enriched to provide predominantly one enantiomer of the lipoic acid salt. An enantiomerically enriched mixture may comprise, for example, at least 60 mol percent of one enantiomer, or more preferably at least 75, 90, 95, or even 99 mol percent. In certain embodiments, the lipoic acid salt is enriched in the (R) enantiomer. In certain embodiments, an (R)-lipoic acid salt is substantially free of the (S)-enantiomer, wherein substantially free means that the substance in question makes up less than 10%, or less than 5%, or less than 4%, or less than 3%, or less than 2%, or less than 1% as compared to the amount of the (R)-enantiomer, e.g., in the composition or compound mixture. For example, if a composition or compound mixture contains 98 grams of the (R)-enantiomer and 2 grams of the (S)-enantiomer, it would be said to contain 98 mol percent of the (R)-enantiomer and only 2% of the (S)-enantiomer. In certain embodiments, the lipoic acid salt is provided as a solvate of the lipoic acid salt.

The compositions described herein have several uses. The present application provides, for example, methods of treating a patient with a NOS-associated disease, such as diabetes, with said compositions. In certain embodiments, the patient suffers from an infection. In certain embodiments, the patient suffers from vasculopathy. In certain embodiments, the patient with diabetes has skin ulcers, including foot ulcers. In certain embodiments, the skin ulcers are treated by topical application of the compositions above. Said compositions may be formulated as a cream. Said compositions may also be included in a wound dressing such as sterile gauze. In alternative embodiments, the compositions are delivered systemically, for example through oral administration, injection, or patch-based delivery.

### BRIEF DESCRIPTION OF THE DRAWINGS

FIG. 1 depicts NMR data showing the ¹H and ¹³C chemical shifts of the lipoic acid salt the compound of Formula III, in _{DMSO-}_{d6}, in graphical and table form.

FIG. 2 depicts the ¹H NMR spectra of the lipoic acid salt of the compound of Formula III, in DMSO-_{d6}.

FIG. 3 depicts the ¹³C NMR spectra of the lipoic acid salt of the compound of Formula III, in DMSO-_{d6}.

FIG. 4 depicts the crystal structure of the lipoic acid salt of the compound of Formula III.

FIG. 5 is a table depicting structural data derived from the crystal structure of the lipoic acid salt of the compound of Formula III.

FIG. 6 is a graph representing the Power XRD pattern of the lipoic acid salt of the compound of Formula III.

FIG. 7 is a chart summarizing the Power XRD pattern of FIG. 6.

FIG. 8 depicts Differential Scanning Calorimetry (DSC) of the lipoic acid salt of the compound of Formula III. The DSC thermogram indicates that: 1. The crystals undergo an endothermic phase transition at 88° C.; 2. The crystals show a sharp melting point at 188.7° C.; and 3. The compound decomposes soon after melting. The decomposition endotherm is broad and spans the temperature range 190-290° C.

FIG. 9 is a graph showing the Thermogravimetric Analysis (TGA) analysis of the lipoic acid salt of the compound of Formula III. The TGA analysis indicates that: 1. In the open pan the complete decomposition of the compound begins at 150° C. and ends at 250° C.; 2. No other transitions were associated with the compound; from this one may infer that there is no solvent loss at all; and 3. The compound totally decomposes by the end of the run within experimental error.

### DETAILED DESCRIPTION

Definitions

As used herein, the following terms and phrases shall have the meanings set forth below. Unless defined otherwise, all technical and scientific terms used herein have the same meaning as commonly understood to one of ordinary skill in the art.

The singular forms “a,” “an,” and “the” include plural reference unless the context clearly dictates otherwise.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acylalkyl” is art-recognized and refers to an alkyl group substituted with an acyl group and may be represented, for example, by the formula hydrocarbylC(O)alkyl.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

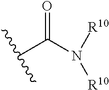

wherein each R¹⁰ independently represent a hydrogen or hydrocarbyl group, or two R¹⁰ are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

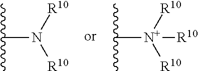

wherein each R¹⁰ independently represents a hydrogen or a hydrocarbyl group, or two R¹⁰ are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

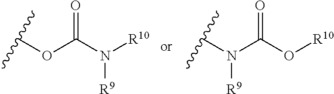

wherein R⁹ and R¹⁰ independently represent hydrogen or a hydrocarbyl group, such as an alkyl group, or R⁹ and R¹⁰ taken together with the intervening atom(s) complete a heterocycle having from 4 to 8 atoms in the ring structure.

The terms “carbocycle”, “carbocyclyl”, and “carbocyclic”, as used herein, refers to a non-aromatic saturated or unsaturated ring in which each atom of the ring is carbon. Preferably a carbocycle ring contains from 3 to 10 atoms, more preferably from 5 to 7 atoms.

The term “carbocyclylalkyl”, as used herein, refers to an alkyl group substituted with a carbocycle group.

The term “carbonate” is art-recognized and refers to a group —OCO₂—R¹⁰, wherein R¹⁰ represents a hydrocarbyl group.

The term “carboxy”, as used herein, refers to a group represented by the formula —CO₂H.

The term “ester”, as used herein, refers to a group -C(O)OR¹⁰ wherein R¹⁰ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “ketone” is art-recognized and may be represented, for example, by the formula —C(O)R₉, wherein R₉ represents a hydrocarbyl group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The term “macromolecule” is art-recognized and refers to a large molecule consisting of many smaller molecules (subunits) linked together. The linkage is typically covalent. Examples of macromolecules include proteins, nucleic acids, complex carbohydrates, and lipids. In certain embodiments, the macromolecule comprises more than 5, 10, 50, 100, 500, or 1000 subunits linked together.

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this application, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

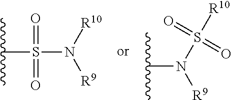

wherein R⁹ and R¹⁰ independently represents hydrogen or hydrocarbyl, such as alkyl, or R⁹ and R¹⁰ taken together with the intervening atom(s) complete a heterocycle having from 4 to 8 atoms in the ring structure.

The term “sulfoxide” is art-recognized and refers to the group —S(O)-R¹⁰, wherein R¹⁰ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R¹⁰ wherein R¹⁰ represents a hydrocarbyl.

The term “thioalkyl”, as used herein, refers to an alkyl group substituted with a thiol group.

The term “thioester”, as used herein, refers to a group —C(O)SR¹⁰ or —SC(O)R¹⁰ wherein R¹⁰ represents a hydrocarbyl.

The term “thioether”, as used herein, is equivalent to an ether, wherein the oxygen is replaced with a sulfur.

The term “thioketone,” as used herein, is equivalent to a ketone, wherein the oxygen is replaced with a sulfur.

The term “urea” is art-recognized and may be represented by the general formula

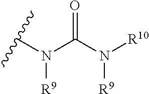

wherein R⁹ and R¹⁰ independently represent hydrogen or a hydrocarbyl, such as alkyl, or either occurrence of R⁹ taken together with R¹⁰ and the intervening atom(s) complete a heterocycle having from 4 to 8 atoms in the ring structure.

The term “diabetes” is used herein to encompass not only an insulin disorder, but any complications arising therefrom. “Diabetes” as used herein encompasses Type I and Type II diabetes; additionally, insulin resistance and defects in insulin production (such as death of pancreatic cells such as pancreatic β cells) are encompassed.

The term “hydrate” as used herein, refers to a compound formed by the union of water with the parent compound.

The term “NOS-associated diseases” as used herein refers to diseases that are associated with elevated NOS levels and/or activity. In certain embodiments, NOS-associated diseases are caused by elevated NOS levels and/or activity. In certain embodiments, NOS-associated diseases are diseases prone to complications that are caused by elevated NOS levels and/or activity. In certain embodiments, the NOS is iNOS.

As used herein, the term “lipoic acid” also encompasses its conjugate base, lipoate. The term “lipoic acid” also includes both stereoisomers (the R and S forms) of lipoic acid and lipoate, as well as all the particular salts of the lipoic acid, such as, for example, the calcium, potassium, magnesium, sodium, or ammonium salt. In certain embodiments, the term “lipoic acid” also encompasses lipoic acid in its free form as well as in a form bound to macromolecules such as the polypeptides ACP, AMP, and an E2 domain containing protein.

The phrases “parenteral administration” and “administered parenterally” are art-recognized terms, and include modes of administration other than enteral and topical administration, such as injections, and include without limitation intravenous, intramuscular, intrapleural, intravascular, intrapericardial, intraarterial, intrathecal, intracapsular, intraorbital, intracardiac, intradennal, intraperitoneal, transtracheal, subcutaneous, subcuticular, intra-articular, subcapsular, subarachnoid, intraspinal and intrastemal injection and infusion.

A “patient,” “subject,” or “host” to be treated by the subject method may mean either a human or non-human animal, such as primates, mammals, and vertebrates.

The phrase “pharmaceutically acceptable” is art-recognized. In certain embodiments, the term includes compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and includes, for example, pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “polymorph” as used herein is art-recognized and refers to one crystal structure of a given compound.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic, (i.e., it is intended to diminish, ameliorate, or stabilize the existing unwanted condition or side effects thereof).

The term “preventing” is art-recognized, and when used in relation to a condition, such as a local effect (e.g., a diabetic foot ulcer), a disease such as Alzheimer's disease, diabetes, or any other medical condition, is well understood in the art, and includes administration of a composition which reduces the frequency of, or delays the onset of, symptoms of a medical condition in a subject relative to a subject which does not receive the composition. Thus, prevention of diabetes includes, for example, reducing the chronically elevated blood glucose levels of a population of patients receiving a prophylactic treatment relative to an untreated control population, and/or delaying the appearance of chronically elevated blood glucose in a treated population versus an untreated control population, e.g., by a statistically and/or clinically significant amount. Prevention of a neurodegenerative disease includes, for example, reducing the number of diagnoses or the severity of the neurodegenerative disease in a treated population versus an untreated control population, and/or delaying the onset of symptoms of the neurodegenerative disease in a treated population versus an untreated control population. Prevention of an inflammatory disease includes, for example, reducing the incidence or severity of, or alternatively delaying, inflammatory disease diagnosed in subjects in a treated population versus an untreated control population.

The term “solvate” as used herein, refers to a compound formed by solvation (e.g., a compound formed by the combination of solvent molecules with molecules or ions of the solute).

When used with respect to a pharmaceutical composition or other material, the term “sustained release” is art-recognized. For example, a subject composition which releases a substance over time may exhibit sustained release characteristics, in contrast to a bolus type administration in which the entire amount of the substance is made biologically available at one time. For example, in particular embodiments, upon contact with body fluids including blood, spinal fluid, mucus secretions, lymph or the like, one or more of the pharmaceutically acceptable excipients may undergo gradual or delayed degradation (e.g., through hydrolysis) with concomitant release of any material incorporated therein, e.g., an therapeutic and/or biologically active salt and/or composition, for a sustained or extended period (as compared to the release from a bolus). This release may result in prolonged delivery of therapeutically effective amounts of any of the therapeutic agents disclosed herein.

The phrases “systemic administration,” “administered systemically,” “peripheral administration” and “administered peripherally” are art-recognized, and include the administration of a subject composition, therapeutic or other material at a site remote from the disease being treated. Administration of an agent directly into, onto, or in the vicinity of a lesion of the disease being treated, even if the agent is subsequently distributed systemically, may be termed “local” or “topical” or “regional” administration, other than directly into the central nervous system, e.g., by subcutaneous administration, such that it enters the patient's system and, thus, is subject to metabolism and other like processes.

As used herein, the term “tautomers” refers to isomeric compounds which differ only in the migration of a proton and movement of a double bond or more than one conjugated double bonds. For example, a compound drawn as Formula I, may exist as its tautomeric forms I or Ia:

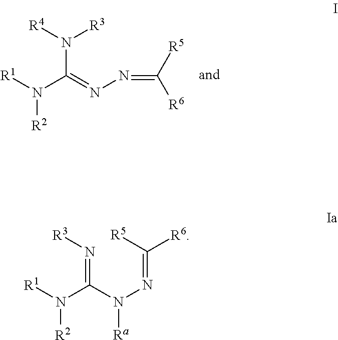

For the purposes of the present application, Formula I should be understood to encompass the tautomers indicated by Formulae I and Ia.

The phrase “therapeutically effective amount” is an art-recognized term. In certain embodiments, the term refers to an amount of a salt or composition disclosed herein that produces some desired effect at a reasonable benefit/risk ratio applicable to any medical treatment. In certain embodiments, the term refers to that amount necessary or sufficient to eliminate or reduce medical symptoms for a period of time. The effective amount may vary depending on such factors as the disease or condition being treated, the particular targeted constructs being administered, the size of the subject, or the severity of the disease or condition. One of ordinary skill in the art may empirically determine the effective amount of a particular composition without necessitating undue experimentation.

The term “treating” is art-recognized and includes preventing a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. The term “treating”, “treat” or “treatment” as used herein includes curative, preventative (e.g., prophylactic), adjunct and palliative treatment.

### 1. Introduction

Aminoguanidine is a known compound Journal of American Chemistry Society 57: 2730 (1935), and is a prototype therapeutic agent for the inhibition of AGE formation Jour. Carbo. Chem., 12(6): 731-742; Diabetes 41:26-29; U.S. Pat. Nos. 5,128,360 and 5,238,963; and is also known to be an inhibitor of NOS, including iNOS Eur. Jour. Pharma., 233, 119-125. However, aminoguanidine has significant safety/tolerability issues that limit its utility. One of the disclosures of the instant application is that the side effects of aminoguanidine may be reduced by co-administration with lipoic acid.

Alpha-lipoic acid has a variety of names. In addition to being known as α-lipoic acid and thioctic acid, it is also known as lipoic acid, 1,2-dithiolane-3-pentanoic acid; 1,2-ditholane-3-valeric acid; 6,8-thioctic acid; 5-[3-C1,2-dithiolanyl)]-pentanoic acid; delta43-(1,2-dithiacyclopentyl)]pentanoic acid; acetate replacing factor and pyruvate oxidation factor. Lipoic acid has an asymmetric carbon atom and is usually employed in the form of a racemic mixture of its (R)- and (S)-enantiomers. It is commercially available (e.g. from Sigma Aldrich). Lipoic acid administration has been shown to be active in oxidative stress models including in ischemia-reperfusion injury model.

2. Lipoic Acid Salts, and Synthesis Thereof

The disclosures herein provide, inter alia, lipoic acid salts of the compounds of Formula I, II, III, or IV, as well as polymorphs, solvates, and hydrates thereof. These salts may be formulated as pharmaceutical compositions. The salts and pharmaceutical compositions may be formulated for oral administration, transdermal administration, or injection. Such compositions may be used to treat NOS-associated diseases such as, for example, inflammatory diseases, metabolic diseases, and neurodegenerative diseases.

The present application also provides a lipoic acid salt of a compound of Formula I:

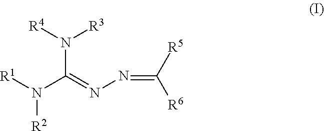

having a lipoate ion enriched for the R-(+) enantiomer, and wherein
R¹ and R², each independently, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, and a macromolecule, or R¹ and R², taken together, form ═C═O, ═CH—CHO, or ═C(R⁷)(R⁸);

R³ and R⁴, each independently, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, and a macromolecule, or R³ and R⁴, taken together, form ═C═O, ═CH—CHO, or ═C(R⁷)(R⁸);

R⁵ and R⁶, each independently, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, thioether, and a macromolecule;

R⁷ and R⁸, each independently, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, thioether, and a macromolecule.

In certain embodiments, R¹ is H, R² is H, R³ is H, R⁴ is H, and
R⁵ and R⁶, each independently, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, thioether, thioketone, and a macromolecule.

In some embodiments, R¹ is H, R² is H, R³ is H, R⁴ is H, R⁵ is CH₃ and R⁶ is CH₃.

In addition, the present application describes the lipoic acid salt a compound of Formula II:

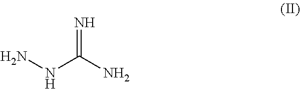

having a lipoate ion enriched for the R-(+) enantiomer.

The disclosures herein also relate to the lipoic acid salt of the compound of Formula III:

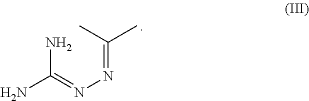

In certain embodiments, the present disclosure provides the lipoic acid salt of a compound of Formula IV:

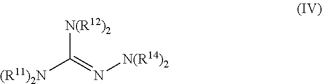

having a lipoate ion enriched for the R-(+) enantiomer, wherein
R¹¹, independently for each occurrence, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, and a macromolecule, or both occurrences of R¹¹ taken together form ═C═O, ═CH—CHO, or ═C(R¹⁵)₂;
R¹², independently for each occurrence, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, and a macromolecule, or both occurrences of R¹² taken together form ═C═O, ═CH—CHO, or ═C(R¹⁵)₂;
R¹⁴, independently for each occurrence, is selected from: H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, and a macromolecule, or both occurrences of R¹⁴ taken together form ═C═O, ═CH—CHO, or ═C(R¹⁵)₂;

R¹⁵, independently for each occurrence, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, and a macromolecule.

In one embodiment, at least one of R¹¹, R¹², and R¹⁴ is other than H.

In certain embodiments, both instances of R¹¹, taken together, are the moiety resulting from the formation of a Schiff base by reaction with a carbonyl, such as aldehydes, ketones, acetone, acetaldehyde, and in general any molecule having a carbonyl moiety. In certain embodiments, both instances of R¹², taken together, are the moiety resulting from the formation of a Schiff base by reaction with a carbonyl, such as aldehydes, ketones, acetone, acetaldehyde, and in general any molecule having a carbonyl moiety. In certain embodiments, both instances of R¹⁴, taken together, are the moiety resulting from the formation of a Schiff base by reaction with a carbonyl, such as aldehydes, ketones, acetone, acetaldehyde, and in general any molecule having a carbonyl moiety.

In certain embodiments, R¹¹ is H, R¹² is H, and
R¹⁴, independently for each occurrence, is selected from: acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, and a macromolecule, or both occurrences of R¹⁴ taken together form ═C═O, ═CH—CHO, or ═C(R¹⁵)₂; and
R¹⁵, independently for each occurrence, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, and a macromolecule.

In certain embodiments, both occurrences of R¹⁴ are methyl.

In certain embodiments, the lower alkyl is a C₁-C₆-alkyl. In certain embodiments, the acyl is a formyl. In certain embodiments, the macromolecule is a polypeptide or oligopeptide. In certain embodiments, the polypeptide is an antibody. In certain embodiments, the salt is in crystalline form. In certain embodiments, the salt is substantially free of the S enantiomer of lipoate.

In certain embodiments, R-(+)-lipoate, which is the conjugate base of R-(+)-lipoic acid, is represented by the molecule below:

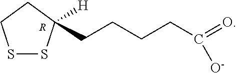

In certain embodiments, the lipoic acid salt of a compound of Formula IV is:

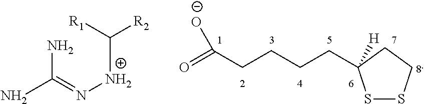

In certain embodiments, the lipoic acid salt of a compound of Formula IV is:

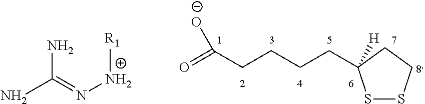

In certain embodiments, the recited structures encompass the tautomers of said structures. For example, the compound of Formula I can form at least the following tautomers:

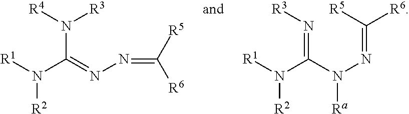

The compound of Formula II can form at least the following tautomers:

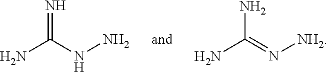

The compound of Formula III can form at least the following tautomers:

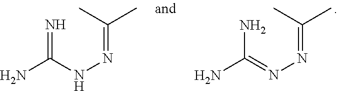

The compounds of Formula IV can form at least the following tautomers:

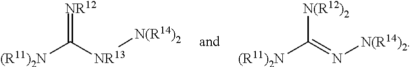

In certain embodiments, the disclosed compounds are charged. In other embodiments, the disclosed compound are uncharged. For example, the compound of Formula III can exist in charged and uncharged forms:

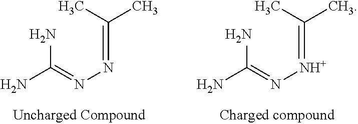

In one aspect, the salts described herein are crystalline because by crystallisation one may obtain a very pure form of the salt. One embodiment envisions the lipoic acid salt a compound of Formula I, II, III, or IV, in the form of a polymorph designated herein after as polymorph A. Polymorph A has high purity and stability, including thermodynamic stability and resistance to moisture in the air (hygroscopicity), as well as high bioavailability. Another advantage of polymorph A is that it is better suitable for the manufacture of pharmaceutical formulations in large scale than said salt in amorphous form because of better handling properties.

The lipoic acid salts of the compounds of the Formulas I, II, III, and IV are readily prepared as set forth below.

A salt of a compound of Formula I, II, III, or IV (for example, aminoguanidine hydrochloride) and lipoic acid may be dissolved in an appropriate inert solvent. As used herein, the expression “inert solvent” refers to a solvent or mixture of solvents, which does not interact with starting materials, reagents, intermediates or products in a manner, which adversely affects the yield of the desired product. Appropriate solvents include methanol, ethanol, n-propanol, isopropanol, butanols, acetonitrile, acetone, ethyl methyl ketone, diethyl ketone and methyl isobutyl ketone. When aminoguanidine salts are employed, a non-reacting base may be used to sufficiently neutralize the salts. Non-reacting bases include alkali and alkali metal hydroxides, alkali and alkali metal carbonates and bicarbonates and tertiary amines. Also included are resin bases. Examples of these non-reacting bases include sodium methoxide, sodium hydroxide, potassium hydroxide, sodium carbonate, potassium carbonate, sodium bicarbonate, potassium bicarbonate, triethyl amine, N-methyl isopropyl amine, and the ion exchange AMBERLYS™ resins.

The reaction mixture is stirred at about ambient temperature to about the refluxing temperature of the solvent being used for about two hours to about six hours, for instance at ambient temperature for about two hours. The reaction mixture may be stirred using any appropriate stirring device. The salts may be isolated from the reaction mixture by methods well known to those skilled in the art and crystallized from an appropriate solvent or mixture of solvents. Alcohols (including methanol), nitriles, acetone are appropriate solvents for crystallization.

The chemist of ordinary skill in the art will also recognize that lipoic acid salts of the compounds of Formula I, II, III, or IV can exist in positional protonated forms, because said compounds contain protonatable nitrogen atoms.

Hydrates and solvates of lipoic acid salts of the compounds of Formulas I, II, III, and IV are also encompassed in the scope of the disclosures herein. Chemists of ordinary skill will also recognize that lipoic acid salts of the compounds of Formulas I, II, III, and IV can exist in different polymorphic forms in the solid state.

Aminoguanidine hydrochloride is prepared as disclosed in Journal of American Chemistry Society 57: 2730 (1935). Lipoic acid is commercially available and its synthesis is reported in, for example, Chem. Commun., 1986, 1408.

### 3. Pharmaceutical Compositions

This application also discloses a pharmaceutical composition comprising a pharmaceutically acceptable carrier and the lipoic acid salt of a compound of Formula I, II, III, or IV. The pharmaceutical composition may be formulated for systemic or topical administration. The pharmaceutical composition may be formulated for oral administration, injection, subdermal administration, or transdermal administration. The pharmaceutical composition may further comprise at least one of a pharmaceutically acceptable stabilizer, diluent, surfactant, filler, binder, and lubricant. The pharmaceutical composition may also include L-arginine.

In most embodiments, the pharmaceutical compositions described herein will incorporate the disclosed salts and compositions (such as lipoic acid salts of the compounds of Formulas I, II, III, and IV) to be delivered in an amount sufficient to deliver to a patient a therapeutically effective amount of a salt and/or composition as part of a prophylactic or therapeutic treatment. The desired concentration of salt and/or composition will depend on absorption, inactivation, and excretion rates of the drug as well as the delivery rate of the salts and compositions from the subject compositions. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

Additionally, the optimal concentration and/or quantities or amounts of any particular salt or composition may be adjusted to accommodate variations in the treatment parameters. Such treatment parameters include the clinical use to which the preparation is put, e.g., the site treated, the type of patient, e.g., human or non-human, adult or child, and the nature of the disease or condition.

The concentration and/or amount of any salt or composition may be readily identified by routine screening in animals, e.g., rats, by screening a range of concentration and/or amounts of the material in question using appropriate assays. Known methods are also available to assay local tissue concentrations, diffusion rates of the salts or compositions, and local blood flow before and after administration of therapeutic formulations disclosed herein. One such method is microdialysis, as reviewed by T. E. Robinson et al., 1991, MICRODIALYSIS IN THE NEUROSCIENCES, Techniques, volume 7, Chapter 1. The methods reviewed by Robinson may be applied, in brief, as follows. A microdialysis loop is placed in situ in a test animal. Dialysis fluid is pumped through the loop. When salts or compositions such as those disclosed herein are injected adjacent to the loop, released drugs are collected in the dialysate in proportion to their local tissue concentrations. The progress of diffusion of the salts or compositions may be determined thereby with suitable calibration procedures using known concentrations of salts or compositions. In the art there are animal model systems for neurodegenerative diseases and inflammatory diseases. Once the correct dosage has been determined in a model system, the correct dose for humans may readily be determined according to Table A:

TABLE A
Conversion of Animal Doses to Human Equivalent Doses (HED)
Based on Body Surface Area (see e.g., Guidance for Industry
Reviewers: Estimating the Safe Starting Dose in Clinical Trials
for Therapeutics in Adult Healthy Volunteers, on the world wide
web at fda.gov/ohrms/dockets/98fr/02d-0492-gdl0001-vol1.pdf).

| | | To convert animal dose in | |
|---|---|---|---|
| | | mg/kg to HED^{a} in | |
| | To convert animal | mg/kg, either: | |
| | dose in mg/kg to dose | | Multiply |
| | in mg/m², multiple by | Divide animal | animal |
| Species | km below: | dose by: | dose by: |
| Human | 37 | — | — |
| Human Child | 25 | — | — |
| (20 kg) | | | |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea Pig | 8 | 4.6 | 0.22 |
| Rabbit | 12 | 3.1 | 0.32 |
| Dog | 20 | 1.8 | 0.54 |
| Monkeys^{b} | 12 | 3.1 | 0.32 |
| Marmoset | 6 | 6.2 | 0.16 |
| Squirrel | 7 | 5.3 | 0.19 |
| Monkey | | | |
| Baboon | 20 | 1.8 | 0.54 |
| Micro-pig | 27 | 1.4 | 0.73 |
| Mini-pig | 35 | 1.1 | 0.95 |

^{a}Assumes 60 kg human. For species not listed or for weights outside the standard ranges, human equivalent dose can be calculated from the formula: HED = animal dose in mg/kg × (animal weight in kg/human weight in kg).
^{b}For example, cynomolgus, rhesus, stumptail.

In certain embodiments, the dosage of the subject salts and compositions provided herein may be determined by reference to the plasma concentrations of the therapeutic composition or other encapsulated materials. For example, the maximum plasma concentration (Cₘₐₓ) and the area under the plasma concentration-time curve from time 0 to infinity may be used.

Generally, in carrying out the methods detailed in this application, an effective dosage for lipoic acid salts of the compounds of Formulas I, II, III, or IV is in the range of about 0.3 mg/kg/day to about 60 mg/kg/day in single or divided doses, for instance 1 mg/kg/day to about 50 mg/kg/day in single or divided doses. When compositions comprising: a) lipoic acid and b) a compound of Formula I, II, III, or IV are used, the dose of the compound of Formula I, II, III, or IV may be in the range 3 mg/kg/day to about 40 mg/kg/day (for instance, 5 mg/kg, 10 mg/kg, 20 mg/kg, or 30 mg/kg), and the dose of lipoic acid may be 5 mg/kg/day to about 70 mg/kg/day (for instance, 5 mg/kg, 10 mg/kg, 20 mg/kg, 30 mg/kg, 40 mg/kg, 50 mg/kg, or 60 mg/kg). When aminoguanidinium and lipoic acid combinations are used, the dose of aminoguanidinium is in the range 3 mg/kg/day to about 40 mg/kg/day (for instance, 5 mg/kg, 10 mg/kg, 20 mg/kg, or 30 mg/kg), and the dose of lipoic acid is 5 mg/kg/day to about 70 mg/kg/day (for instance, 5 mg/kg, 10 mg/kg, 20 mg/kg, 30 mg/kg, 40 mg/kg, 50 mg/kg, or 60 mg/kg).

An effective amount of the salts and compositions described herein refers to the amount of one of said salts or compositions which is capable of inhibiting or preventing a disease. This disease may be, for example, a NOS-associated disease including an inflammatory or neurodegenerative disease. This disease may be diabetic complications and/or, type 1 or type 2 diabetes. An effective amount may be sufficient to prohibit, treat, alleviate, ameliorate, halt, restrain, slow or reverse the progression, or reduce the severity of a complication resulting from elevated advanced glycation end products (AGE) and/or elevated reactive oxidative-nitrosative species and/or elevated nitric oxide synthase (NOS) activity, in patients who are at risk for such complications. As such, these methods include both medical therapeutic (acute) and/or prophylactic (prevention) administration as appropriate. The amount and timing of compositions administered will, of course, be dependent on the subject being treated, on the severity of the affliction, on the manner of administration and on the judgment of the prescribing physician. Thus, because of patient-to-patient variability, the dosages given above are a guideline and the physician may titrate doses of the drug to achieve the treatment that the physician considers appropriate for the patient. In considering the degree of treatment desired, the physician must balance a variety of factors such as age of the patient, presence of preexisting disease, as well as presence of other diseases.

The compositions provided by this application may be administered to a subject in need of treatment by a variety of conventional routes of administration, including orally, topically, parenterally, e.g., intravenously, subcutaneously or intramedullary. Further, the compositions may be administered intranasally, as a rectal suppository, or using a “flash” formulation, i.e., allowing the medication to dissolve in the mouth without the need to use water. Furthermore, the compositions may be administered to a subject in need of treatment by controlled release dosage forms, site specific drug delivery, transdermal drug delivery, patch (active/passive) mediated drug delivery, by stereotactic injection, or in nanoparticles.

The compositions may be administered alone or in combination with pharmaceutically acceptable carriers, vehicles or diluents, in either single or multiple doses. Suitable pharmaceutical carriers, vehicles and diluents include inert solid diluents or fillers, sterile aqueous solutions and various organic solvents. The pharmaceutical compositions formed by combining the compositions and the pharmaceutically acceptable carriers, vehicles or diluents are then readily administered in a variety of dosage forms such as tablets, powders, lozenges, syrups, injectable solutions and the like. These pharmaceutical compositions can, if desired, contain additional ingredients such as flavorings, binders, excipients and the like. Thus, for purposes of oral administration, tablets containing various excipients such as L-arginine, sodium citrate, calcium carbonate and calcium phosphate may be employed along with various disintegrates such as starch, alginic acid and certain complex silicates, together with binding agents such as polyvinylpyrrolidone, sucrose, gelatin and acacia. Additionally, lubricating agents such as magnesium stearate, sodium lauryl sulfate and talc are often useful for tabletting purposes. Solid compositions of a similar type may also be employed as fillers in soft and hard filled gelatin capsules. Appropriate materials for this include lactose or milk sugar and high molecular weight polyethylene glycols. When aqueous suspensions or elixirs are desired for oral administration, the essential active ingredient therein may be combined with various sweetening or flavoring agents, coloring matter or dyes and, if desired, emulsifying or suspending agents, together with diluents such as water, ethanol, propylene glycol, glycerin and combinations thereof.

This application provides, inter alia, certain compositions comprising lipoic acid (or lipoate), compounds of Formulas I, II, III, or IV (or their conjugate acids), and amino acids such as L-arginine. In certain embodiments, the L-arginine is substantially free of R-arginine. In certain embodiments, the lipoic acid is R-(+)-lipoic acid.

Among other things, the present application discloses a composition comprising the lipoic acid salt of the compound of Formula I, II, III, or IV, and an amino acid. In a preferred embodiment, the amino acid is L-arginine. Herein applicants also disclose a composition comprising the lipoic acid salt of the compound of Formula I, II, III, or IV, and an amino acid such as L-arginine.

Additionally, herein Applicants provide composition comprising the lipoic acid salt of an amino acid (or the conjugate base of said amino acid), and the compound of Formula I, II, III, of IV. In some embodiments, the amino acid is L-arginine.

Furthermore, the present application also provides a composition comprising: a) a lipoic acid salt of an amino acid, or the conjugate base of said amino acid, and b) a lipoic acid salt of the compound of Formula I, II, III, or IV. In some embodiments, the amino acid is L-arginine. According to a non-limiting theory herein, L-arginine may improve the bioavailability of other components of the composition.

For parenteral administration, solutions of the compositions may be prepared in sesame or peanut oil, aqueous propylene glycol, or in sterile aqueous solutions may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, the sterile aqueous media employed are all readily available by standard techniques known to those skilled in the art.

The formulations, for instance tablets, may contain e.g. 3 to 800, or 20 to 600, e.g. 50, 250, 300, or 400, mg of the salts and compositions disclosed herein, for instance lipoic acid salts of the compounds of Formulas I, II, III, or IV.

Generally, a composition as described herein may be administered orally, or parenterally (e.g., intravenous, intramuscular, subcutaneous or intramedullary). Topical administration may also be indicated, for example, where the patient is suffering from gastrointestinal disorder that prevent oral administration, or whenever the medication is best applied to the surface of a tissue or organ as determined by the attending physician. Localized administration may also be indicated, for example, when a high dose is desired at the target tissue or organ. For buccal administration the active composition may take the form of tablets or lozenges formulated in a conventional manner.

For purposes of transdermal (e.g., topical) administration, dilute sterile, aqueous or partially aqueous solutions (usually in about 0.1% to 5% concentration), otherwise similar to the above parenteral solutions, may be prepared.

Methods of preparing various pharmaceutical compositions with a certain amount of one or more salts or other active agents are known, or will be apparent in light of this disclosure, to those skilled in this art. For examples of methods of preparing pharmaceutical compositions, see Remington's Pharmaceutical Sciences, Mack Publishing Company, Easton, Pa., 19th Edition (1995).

The compositions described herein may be administered by various means, depending on their intended use, as is well known in the art. For example, if subject compositions are to be administered orally, it may be formulated as tablets, capsules, granules, powders or syrups. Alternatively, formulations described herein may be administered parenterally as injections (intravenous, intramuscular, or subcutaneous), drop infusion preparations, or suppositories. For application by the ophthalmic mucous membrane route, subject compositions may be formulated as eyedrops or eye ointments. These formulations may be prepared by conventional means, and, if desired, the subject compositions may be mixed with any conventional additive, such as a binder, a disintegrating agent, a lubricant, a corrigent, a solubilizing agent, a suspension aid, an emulsifying agent or a coating agent.

In addition, in certain embodiments, subject compositions of the present application maybe lyophilized or subjected to another appropriate drying technique such as spray drying.

The subject compositions may be administered once, or may be divided into a number of smaller doses to be administered at varying intervals of time, depending in part on the release rate of the compositions and the desired dosage.

Formulations useful in the methods provided herein include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of a subject composition which may be combined with a carrier material to produce a single dose may vary depending upon the subject being treated, and the particular mode of administration.

Methods of preparing these formulations or compositions include the step of bringing into association subject compositions with the carrier and, optionally, one or more accessory ingredients. In general, the formulations are prepared by uniformly and intimately bringing into association a subject composition with liquid carriers, or finely divided solid carriers, or both, and then, if necessary, shaping the product.

The salts and compositions described herein may be administered in inhalant or aerosol formulations. The inhalant or aerosol formulations may comprise one or more agents, such as adjuvants, diagnostic agents, imaging agents, or therapeutic agents useful in inhalation therapy. The final aerosol formulation may for example contain 0.005-90% w/w, for instance 0.005-50%, 0.005-5% w/w, or 0.01-1.0% w/w, of medicament relative to the total weight of the formulation.

It is desirable, but by no means required, that the formulations herein contain no components which may provoke the degradation of stratospheric ozone. In particular it is desirable that the formulations are substantially free of chlorofluorocarbons such as CCl₃F, CCl₂F₂ and CF₃CCl₃. As used to refer to ozone-damaging agents, “substantially free” means less than 1% w/w based upon the propellant system, in particular less than 0.5%, for example 0.1% or less.

The propellant may optionally contain an adjuvant having a higher polarity and/or a higher boiling point than the propellant. Polar adjuvants which may be used include (e.g., C₂₋₆) aliphatic alcohols and polyols such as ethanol, isopropanol and propylene glycol. In general, only small quantities of polar adjuvants (e.g., 0.05-3.0% w/w) may be required to improve the stability of the dispersion--the use of quantities in excess of 5% w/w may tend to dissolve the medicament. The formulations described herein may contain less than 1% w/w, e.g., about 0.1% w/w, of polar adjuvant. However, the formulations may be substantially free of polar adjuvants, such as ethanol. Suitable volatile adjuvants include saturated hydrocarbons such as propane, n-butane, isobutane, pentane and isopentane and alkyl ethers such as dimethyl ether. In general, up to 50% w/w of the propellant may comprise a volatile adjuvant, for example 1 to 30% w/w of a volatile saturated C₁-C₆ hydrocarbon.

Optionally, the aerosol formulations may further comprise one or more surfactants. The surfactants must be physiologically acceptable upon administration by inhalation. Within this category are included surfactants such as L-α-phosphatidylcholine (PC), 1,2-dipalmitoylphosphatidycholine (DPPC), oleic acid, sorbitan trioleate, sorbitan mono-oleate, sorbitan monolaurate, polyoxyethylene (20) sorbitan monolaurate, polyoxyethylene (20) sorbitan monooleate, natural lecithin, oleyl polyoxyethylene (2) ether, stearyl polyoxyethylene (2) ether, lauryl polyoxyethylene (4) ether, block copolymers of oxyethylene and oxypropylene, synthetic lecithin, diethylene glycol dioleate, tetrahydrofurfuryl oleate, ethyl oleate, isopropyl myristate, glyceryl monooleate, glyceryl monostearate, glyceryl monoricinoleate, cetyl alcohol, stearyl alcohol, polyethylene glycol 400, cetyl pyridinium chloride, benzalkonium chloride, olive oil, glyceryl monolaurate, corn oil, cotton seed oil, and sunflower seed oil. Appropriate surfactants include lecithin, oleic acid, and sorbitan trioleate.

Ophthalmic formulations, eye ointments, powders, solutions and the like, are also contemplated as being within the scope of the disclosures herein.

Certain pharmaceutical compositions disclosed herein suitable for parenteral administration comprise one or more subject compositions in combination with one or more pharmaceutically acceptable sterile, isotonic, aqueous, or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition as an active ingredient. Subject compositions may also be administered as a bolus, electuary, or paste.

In solid dosage forms for oral administration (capsules, tablets, pills, dragees, powders, granules and the like), the subject composition is mixed with one or more pharmaceutically acceptable carriers and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using lactose or milk sugars, as well as high molecular weight polyethylene glycols and the like.

A tablet may be made by compression or molding, optionally with one or more accessory ingredients. Compressed tablets may be prepared using a binder (for example, gelatin or hydroxypropylmethyl cellulose), lubricant, inert diluent, preservative, disintegrant (for example, sodium starch glycolate or cross-linked sodium carboxymethyl cellulose), surface-altering or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

There has been widespread use of tablets since the latter part of the 19^{th} century and the majority of pharmaceutical dosage forms are marketed as tablets. Major reasons of tablet popularity as a dosage form are simplicity, low cost and the speed of production. Other reasons include stability of drug product, convenience in packaging, shipping and dispensing. To the patient or consumer, tablets offer convenience of administration, ease of accurate dosage, compactness, portability, blandness of taste, ease of administration and elegant distinctive appearance.

Tablets may be plain, film or sugar coated, bisected, embossed, layered or sustained-release. They can be made in a variety of sizes, shapes and colors. Tablets may be swallowed, chewed or dissolved in the buccal cavity or beneath the tongue. They may be dissolved in water for local or topical application. Sterile tablets are normally used for parenteral solutions and for implantation beneath the skin.

In addition to the active or therapeutic ingredients, tablets may contain a number of inert materials known as excipients. They may be classified according to the role they play in the final tablet. The primary composition may include one or more of a filler, binder, lubricant and glidant. Other excipients which give physical characteristics to the finished tablet are coloring agents, and flavors (especially in the case of chewable tablets). Without excipients most drugs and pharmaceutical ingredients cannot be directly-compressed into tablets. This is primarily due to the poor flow and cohesive properties of most drugs. Typically, excipients are added to a formulation to impart good flow and compression characteristics to the material being compressed. Such properties are imparted through pretreatment steps, such as wet granulation, slugging, spray drying spheronization or crystallization.

Lubricants are typically added to prevent the tableting materials from sticking to punches, minimize friction during tablet compression, and allow for removal of the compressed tablet from the die. Such lubricants are commonly included in the final tablet mix in amounts usually of about 1% by weight.

Other desirable characteristics of excipients include the following: high-compressibility to allow strong tablets to be made at low compression forces; impart cohesive qualities to the powdered material; acceptable rate of disintegration; good flow properties that can improve the flow of other excipients in the formula; and cohesiveness (to prevent tablet from crumbling during processing, shipping and handling).

There are at least three commercially important processes for making compressed tablets: wet granulation, direct compression and dry granulation (slugging or roller compaction). The method of preparation and type of excipients are selected to give the tablet formulation the desired physical characteristics that allow for the rapid compression of the tablets. After compression, the tablets must have a number of additional attributes, such as appearance, hardness, disintegrating ability and an acceptable dissolution profile. Choice of fillers and other excipients will depend on the chemical and physical properties of the drug, behavior of the mixture during processing and the properties of the final tablets. Preformulation studies are done to determine the chemical and physical compatibility of the active component with proposed excipients.

The properties of the drug, its dosage forms and the economics of the operation will determine selection of the best process for tableting. Generally, both wet granulation and direct compression are used in developing a tablet.

One formulation comprises the following: the lipoic acid salt of a compound of Formula I, II, III, or IV, and a binder. Examples of pharmaceutically acceptable binders include, but are not limited to, starches; celluloses and derivatives thereof, e.g., microcrystalline cellulose, hydroxypropyl cellulose hydroxylethyl cellulose and hydroxylpropylmethyl cellulose; sucrose; dextrose; corn syrup; polysaccharides; and gelatin. The binder, e.g., may be present in an amount from about 1% to about 40% by weight of the composition such as 1% to 30% or 1% to 25% or 1% to 20%.

Optionally, one, two, three or more diluents can be added to the formulations disclosed herein. Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc. The filler and/or diluent, e.g., may be present in an amount from about 15% to about 40% by weight of the composition. In certain embodiments, diluents are microcrystalline cellulose which is manufactured by the controlled hydrolysis of alpha-cellulose, obtained as a pulp from fibrous plant materials, with dilute mineral acid solutions. Following hydrolysis, the hydrocellulose is purified by filtration and the aqueous slurry is spray dried to form dry, porous particles of a broad size distribution. Suitable microcrystalline cellulose will have an average particle size of from about 20 nm to about 200 nm. Microcrystalline cellulose is available from several suppliers. Suitable microcrystalline cellulose includes Avicel PH 101 , Avicel PH 102, Avicel PH 103, Avicel PH 105 and Avicel PH 200, manufactured by FMC Corporation. The microcrystalline cellulose may be present in a tablet formulation in an amount of from about 25% to about 70% by weight. Another appropriate range of this material is from about 30% to about 35% by weight; yet another appropriate range of from about 30% to about 32% by weight. Another diluent is lactose. The lactose may be ground to have an average particle size of between about 50 μm and about 500 μm prior to formulating. The lactose may be present in the tablet formulation in an amount of from about 5% to about 40% by weight, and can be from about 18% to about 35% by weight, for example, can be from about 20% to about 25% by weight.

Optionally one, two, three or more disintegrants can be added to the formulations described herein. Examples of pharmaceutically acceptable disintegrants include, but are not limited to, starches; clays; celluloses; alginates; gums; cross-linked polymers, e.g., cross-linked polyvinyl pyrrolidone, cross-linked calcium carboxymethylcellulose and cross-linked sodium carboxymethylcellulose; soy polysaccharides; and guar gum. The disintegrant, e.g., may be present in an amount from about 2% to about 20%, e.g., from about 5% to about 10%, e.g., about 7% about by weight of the composition. A disintegrant is also an optional but useful component of the tablet formulation. Disintegrants are included to ensure that the tablet has an acceptable rate of disintegration. Typical disintegrants include starch derivatives and salts of carboxymethylcellulose. Sodium starch glycolate is one appropriate disintegrant for this formulation. In certain embodiments, the disintegrant is present in the tablet formulation in an amount of from about 0% to about 10% by weight, and can be from about 1% to about 4% by weight, for instance from about 1.5% to about 2.5% by weight.

Optionally one, two, three or more lubricants can be added to the formulations disclosed herein. Examples of pharmaceutically acceptable lubricants and pharmaceutically acceptable glidants include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose and microcrystalline cellulose. The lubricant, e.g., may be present in an amount from about 0.1% to about 5% by weight of the composition; whereas, the glidant, e.g., may be present in an amount from about 0.1% to about 10% by weight. Lubricants are typically added to prevent the tableting materials from sticking to punches, minimize friction during tablet compression and allow for removal of the compressed tablet from the die. Such lubricants are commonly included in the final tablet mix in amounts usually less than 1% by weight. The lubricant component may be hydrophobic or hydrophilic. Examples of such lubricants include stearic acid, talc and magnesium stearate. Magnesium stearate reduces the friction between the die wall and tablet mix during the compression and ejection of the tablets. It helps prevent adhesion of tablets to the punches and dies. Magnesium stearate also aids in the flow of the powder in the hopper and into the die. It has a particle size range of 450-550 microns and a density range of 1.00-1.80 g/mL It is stable and does not polymerize within the tableting mix. One lubricant, magnesium stearate may also be employed in the formulation. In some aspects, the lubricant is present in the tablet formulation in an amount of from about 0.25% to about 6%; also appropriate is a level of about 0.5% to about 4% by weight; and from about 0.1% to about 2% by weight. Other possible lubricants include talc, polyethylene glycol, silica and hardened vegetable oils. In an optional embodiment, the lubricant is not present in the formulation, but is sprayed onto the dies or the punches rather than being added directly to the formulation.

Other conventional solid fillers or carriers, such as, cornstarch, calcium phosphate, calcium sulfate, calcium stearate, magnesium stearate, stearic acid, glyceryl mono- and distearate, sorbitol, mannitol, gelatin, natural or synthetic gums, such as carboxymethyl cellulose, methyl cellulose, alginate, dextran, acacia gum, karaya gum, locust bean gum, tragacanth and the like, diluents, binders, lubricants, disintegrators, coloring and flavoring agents could optionally be employed.

Additional examples of useful excipients which can optionally be added to the composition are described in the Handbook of Pharmaceutical Excipients, 3rd edition , Edited by A. H. Kibbe, Published by: American Pharmaceutical Association, Washington D.C., ISBN: 0-917330-96-X, or Handbook of Pharmaceutical Excipients (4^{th} edition), Edited by Raymond C Rowe—Publisher: Science and Practice.

Liquid dosage forms for oral administration include pharmaceutically acceptable emulsions, microemulsions, solutions, suspensions, syrups and elixirs. In addition to the subject compositions, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, corn, peanut, sunflower, soybean, olive, castor, and sesame oils), glycerol, tetrahydrofuryl alcohol, polyethylene glycols and fatty acid esters of sorbitan, and mixtures thereof.

Suspensions, in addition to the subject compositions, may contain suspending agents such as, for example, ethoxylated isostearyl alcohols, polyoxyethylene sorbitol, and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating carriers comprising, for example, cocoa butter, polyethylene glycol, a suppository wax, or a salicylate, and which is solid at room temperature, but liquid at body temperature and, therefore, will melt in the appropriate body cavity and release the encapsulated salt(s) and composition(s). Formulations which are suitable for vaginal administration also include pessaries, tampons, creams, gels, pastes, foams, or spray formulations containing such carriers as are known in the art to be appropriate.

Dosage forms for transdermal administration include powders, sprays, ointments, pastes, creams, lotions, gels, solutions, patches, and inhalants. A subject composition may be mixed under sterile conditions with a pharmaceutically acceptable carrier, and with any preservatives, buffers, or propellants that may be required. For transdermal administration, the complexes may include lipophilic and hydrophilic groups to achieve the desired water solubility and transport properties.

The ointments, pastes, creams and gels may contain, in addition to subject compositions, other carriers, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays may contain, in addition to a subject composition, excipients such as lactose, talc, silicic acid, aluminum hydroxide, calcium silicates and polyamide powder, or mixtures of such substances. Sprays may additionally contain customary propellants, such as chlorofluorohydrocarbons and volatile unsubstituted hydrocarbons, such as butane and propane.

Methods of delivering a composition or compositions via a transdermal patch are known in the art. Exemplary patches and methods of patch delivery are described in U.S. Pat. Nos. 6,974,588, 6,564,093, 6,312,716, 6,440,454, 6,267,983, 6,239,180, and 6,103,275.

In one embodiment, a transdermal patch may comprise an outer backing foil, a matrix and a protective liner wherein a) the composition or compositions are present in the matrix in a solution (which may be oversaturated), b) the matrix may contain 1 to 5% activated SiO₂, and c) the matrix may have a moisture content of less than 0.7%. Moisture-free matrix patches which contain activated silicon dioxide in the matrix show an enhanced drug release into the skin.

In another embodiment, a transdermal patch may comprise: a substrate sheet comprising a composite film formed of a resin composition comprising 100 parts by weight of a polyvinyl chloride-polyurethane composite and 2-10 parts by weight of a styrene-ethylene-butylene-styrene copolymer, a first adhesive layer on the one side of the composite film, and a polyalkylene terephthalate film adhered to the one side of the composite film by means of the first adhesive layer, a primer layer which comprises a saturated polyester resin and is formed on the surface of the polyalkylene terephthalate film; and a second adhesive layer comprising a styrene-diene-styrene block copolymer containing a pharmaceutical agent layered on the primer layer. A method for the manufacture of the above-mentioned substrate sheet comprises preparing the above resin composition molding the resin composition into a composite film by a calendar process, and then adhering a polyalkylene terephthalate film on one side of the composite film by means of an adhesive layer thereby forming the substrate sheet, and forming a primer layer comprising a saturated polyester resin on the outer surface of the polyalkylene terephthalate film.

The pharmaceutical compositions herein can be packaged to produce a “reservoir type” transdermal patch with or without a rate-limiting patch membrane. The size of the patch and or the rate limiting membrane can be chosen to deliver the transdermal flux rates desired. Such a transdermal patch can consist of a polypropylene/polyester impervious backing member heat-sealed to a polypropylene porous/permeable membrane with a reservoir therebetween. The patch can include a pharmaceutically acceptable adhesive (such as a acrylate, silicone or rubber adhesive) on the membrane layer to adhere the patch to the skin of the host, e.g., a mammal such as a human. A release liner such as a polyester release liner can also be provided to cover the adhesive layer prior to application of the patch to the skin as is conventional in the art. This patch assembly can be packaged in an aluminum foil or other suitable pouch, again as is conventional in the art.

Alternatively, the compositions herein can be formulated into a “matrix-type” transdermal patch. Drug Delivery Systems Characteristics and Biomedical Application, R. L Juliano, ed., Oxford University Press. N.Y. (1980); and Controlled Drug Delivery, Vol. I Basic Concepts, Stephen D. Bruck (1983) describe the theory and application of methods useful for transdermal delivery systems. The drug-matrix could be formed utilizing various polymers, e.g. silicone, polyvinyl alcohol. The “drug matrix” may then be packaged into an appropriate transdermal patch.

Another type of patch comprises incorporating the drug directly in a pharmaceutically acceptable adhesive and laminating the drug-containing adhesive onto a suitable backing member, e.g. a polyester backing membrane. The drug should be present at a concentration which will not affect the adhesive properties, and at the same time deliver the required clinical dose.

Transdermal patches may be passive or active. Passive transdermal drug delivery systems currently available, such as the nicotine, estrogen and nitroglycerine patches, deliver small-molecule drugs. Many of the newly developed proteins and peptide drugs are too large to be delivered through passive transdermal patches and may be delivered using technology such as electrical assist (iontophoresis) for large-molecule drugs.

Iontophoresis is a technique employed for enhancing the flux of ionized substances through membranes by application of electric current. One example of an iontophoretic membrane is given in U.S. Pat. No. 5,080,646 to Theeuwes. The principal mechanisms by which iontophoresis enhances molecular transport across the skin are (a) repelling a charged ion from an electrode of the same charge, (b) electroosmosis, the convective movement of solvent that occurs through a charged pore in response the preferential passage of counter-ions when an electric field is applied or (c) increase skin permeability due to application of electrical current.

In some cases, it may be desirable to administer two pharmaceutical compositions separately to a patient. Therefore, the present application discloses, inter alia, a kit that comprises two separate pharmaceutical compositions: 1) (R)-(+)-lipoic acid or a pharmaceutically acceptable salt thereof; and 2) a second pharmaceutical composition that is the compound of Formula I, II, III, or IV or a prodrug thereof or a pharmaceutically acceptable salt of the composition or prodrug. The kit may comprise a container for containing the separate compositions such as a divided bottle or a divided foil packet. Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are widely used for the packaging of pharmaceutical unit dosage forms (tablets, capsules, and the like). Blister packs generally consist of a sheet of relatively stiff material covered with a foil of a plastic material that may be transparent. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. In some embodiments the strength of the sheet is such that the tablets or capsules can be removed from the blister pack by manually applying pressure on the recesses whereby an opening is formed in the sheet at the place of the recess. The tablet or capsule can then be removed via said opening.

In certain embodiments, the compositions described herein are prodrugs. For example, according to the non-limiting theory herein, when ingested, the compound of Formula I, III or IV may be converted to the compound of Formula II.

### 4. Methods of Treating NOS-Associated Disorders

The present application discloses, inter alia, methods of treating NOS-associated disorders, comprising administering to a patient in need thereof, a therapeutically effective amount of a lipoic acid salt. NOS-associated diseases include inflammatory diseases, neurodegenerative diseases, and metabolic diseases.

Examples of neurodegenerative diseases treatable by the salts, compositions, and methods herein include demyelinating diseases, including multiple sclerosis; Alzheimer's disease; Pick's disease; Parkinsonism; idiopathic Parkinson disease (paralysis agitans); Huntington disease; degenerative diseases affecting motor neurons, including amyotrophic lateral sclerosis, bulbospinal atrophy (Kennedy syndrome), and spinal muscular atrophy; inborn errors of metabolism, such as leukodystrophies, including Krabbe's disease; and HIV-associated dementia.

Examples of inflammatory diseases treatable as described herein include without limitation: chronic inflammatory disorders of the joints, such as arthritis, rheumatoid arthritis, juvenile idiopathic arthritis, psoriatic arthritis, and osteoarthritis; inflammatory bowel diseases, such as ulcerative colitis and Crohn's disease; inflammatory lung disorders, such as asthma; inflammatory diseases of the kidney, such as uremic complications, glomerulonephritis and nephrosis; inflammatory disorders of the skin, such as sclerodermatitis, psoriasis, erythema, eczema, or contact dermatitis; systemic lupus erythematosus (SLE); inflammatory diseases of the heart, such as cardiomyopathy, ischemic heart disease, hypercholesterolemia, and atherosclerosis. Inflammatory diseases treatable as described herein further include systemic inflammations of the body such as those produced by infection and sepsis.

Metabolic diseases treatable as described herein include diabetes mellitus (also called diabetes), cardio-metabolic syndrome (a high risk of developing a full type 2 diabetes), and diabetic complications. Diabetic complications include microalbuminuria; proteinuria; hypertension; micro-angiopathy comprising nephropathy (glomerulosclerosis, albuminuria), retinopathy (microaneurysm, vascular sclerosis, pupille oedema, proliferative retinopathy, and cataracts), arteriolosclerosis (peripheral circulatory diseases), diabetic ulcers including diabetic foot ulcers, diabetic neuropathy and peripheral neuropathy (polynevritis); macro-angiopathy and atherosclerosis comprising coronary disease, myocardial ischemia, angor pectoris, stroke, cerebrovascular disease, myocardial infarction, and peripheral vascular disease (intermittent claudication); diabetic cataracts; and diabetic neovascular glaucoma.

Some neurodegenerative diseases may have inflammatory components (such as multiple sclerosis), and some inflammatory diseases may have deleterious effects on the nervous system (such as diabetes), so these designations are not mutually exclusive. In certain embodiments, an inflammatory disease is a disease in which the immune system is inappropriately activated. Methods of detecting and diagnosing inflammatory diseases are known in the art. For instance, inappropriate immune system activity may be detected by measuring the levels of autoantibodies in the blood. In some embodiments, a neurodegenerative disease is a disease in which there is progressive neuron death. Methods of detecting and diagnosing neurodegenerative disorders are also known in the art. Said methods may include behavioral or cognitive tests and CAT, MRI, EEG, PET, SPECT, and MRSI scans.

### EXAMPLES

### Example 1

### Preparation of a the Lipoic Acid Salt of the Compound of Formula III

Sodium methoxide (2.4 gms) was dissolved in 10 mL methanol and to this solution was added 5.0 g Aminoguanidine hydrochloride while stirring. The stirring was continued for an additional 20 min. 200 mL acetone was then added, stirred for 30 min, and the mixture was filtered. To the filtrate, containing aminoguanidine in the form of its free base, 9.3 g R-(+)-lipoic acid dissolved in 100 mL acetone was added drop wise with constant stirring resulting in the precipitation of a pale yellow solid. The mixture was stirred for an additional 20 min. and filtered. The light yellow solid was washed with 30 mL acetone, filtered, and dried to yield the lipoic acid salt of the compound of Formula III (yield: 95%).

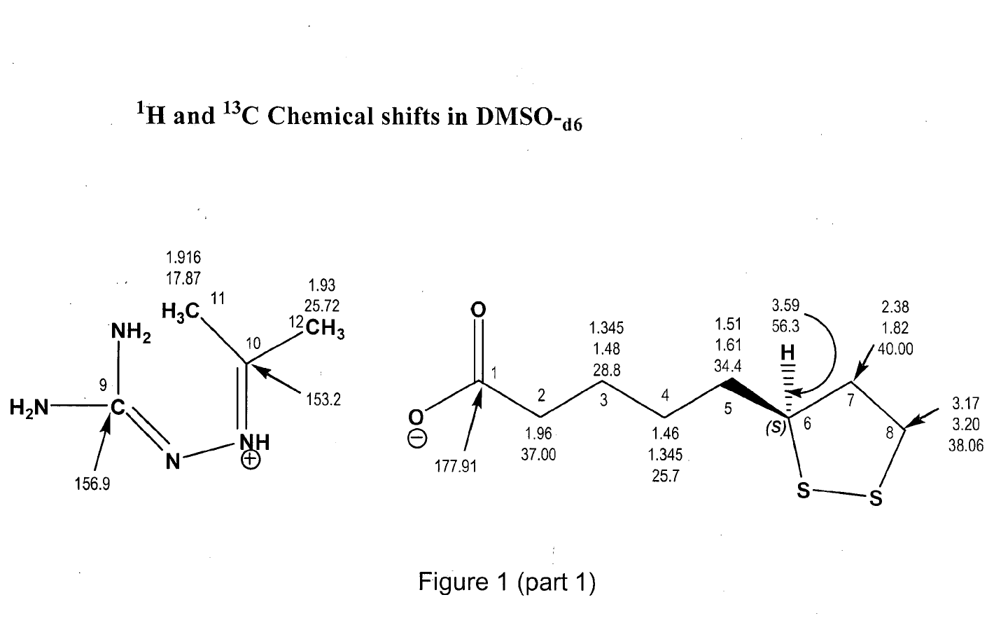

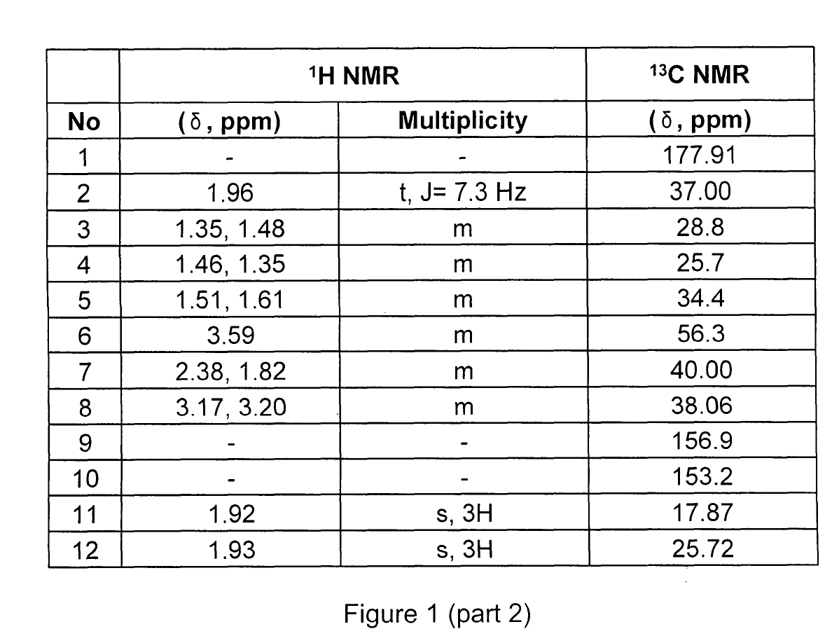

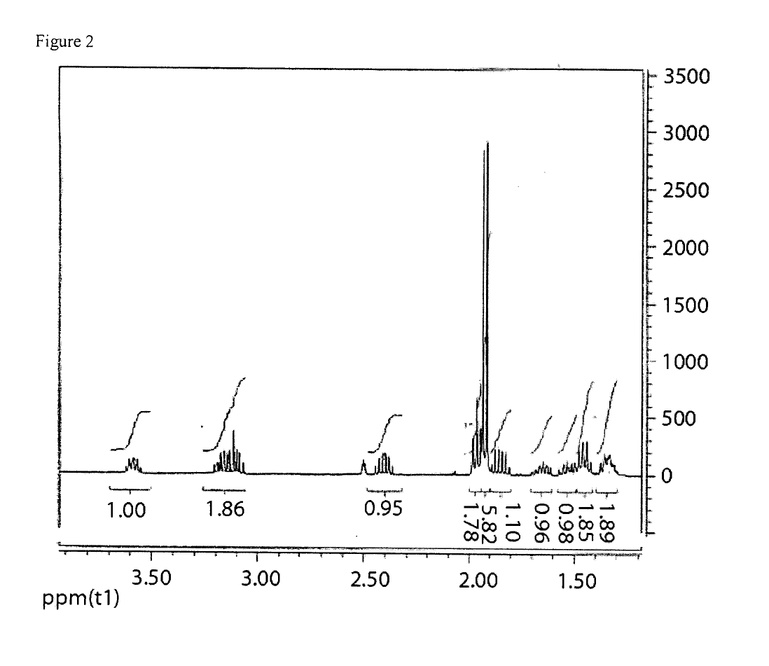

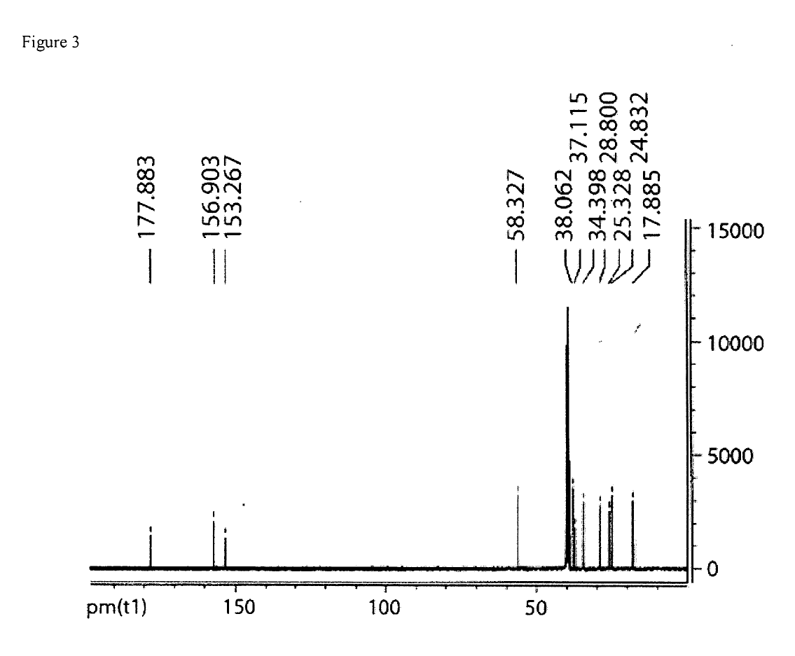

FIGS. 1 through 3 illustrate NMR data used to identify the resulting salt as the lipoic acid salt of the compound of Formula III.

Liquid chromatography-mass spectrometry (LC-MS) was also used to identify the resulting salt as the lipoic acid salt of the compound of Formula III. LC-MS showed two peaks corresponding to:
R-(+)-alpha lipoic acid: Retention time 10.59 min, Molecular weight 207 (m+H)
Schiff's base adduct of aminoguanidine: Retention time 1.20 min, Molecular weight 114 (m+H)

Furthermore, the optical rotation of the resulting salt was determined to be [α]_{D}²⁵=64.5 to 67.5 (c=1, methanol).

The melting point of the resulting salt was determined to be 163° C.-176° C.

### Example 2

### Recrystallization of the Lipoic Acid Salt of the Compound of Formula III

The salt produced in Example 1 (50 mg) was dissolved in 1:1 water-Methanol (800 ul) with stirring and heating in water bath at 60° C. This solution was centrifuged for 10 min. Acetonitrile was added slowly to the supernatant with a continuous vortex. Acetonitrile (˜20 mL) was added until a semi-permanent turbidity turns to a clear solution by stirring. The solution was left undisturbed in the refrigerator at 4° C. for 10-15 hours. Fine crystalline needles are formed, they are filtered and dried.

In addition, CHN analysis was performed. The theoretical values were determined to be: % C 44.97; % H 7.55; % N 17.48; % S 20.01. The experimental values were determined to be:
Salt: % C 44.63; % H 7.57; % N 17.36; % S 20.06
Re-crystallized salt: % C 44.96; % H 7.70; % N 17.43; % S 20.21

The re-crystallized salt was found to have the same melting point, optical rotation, and NMR spectra as the salt prior to re-crystallization.

### Example 3

### Crystal Structure of the Lipoic Acid Salt of the Compound of Formula III

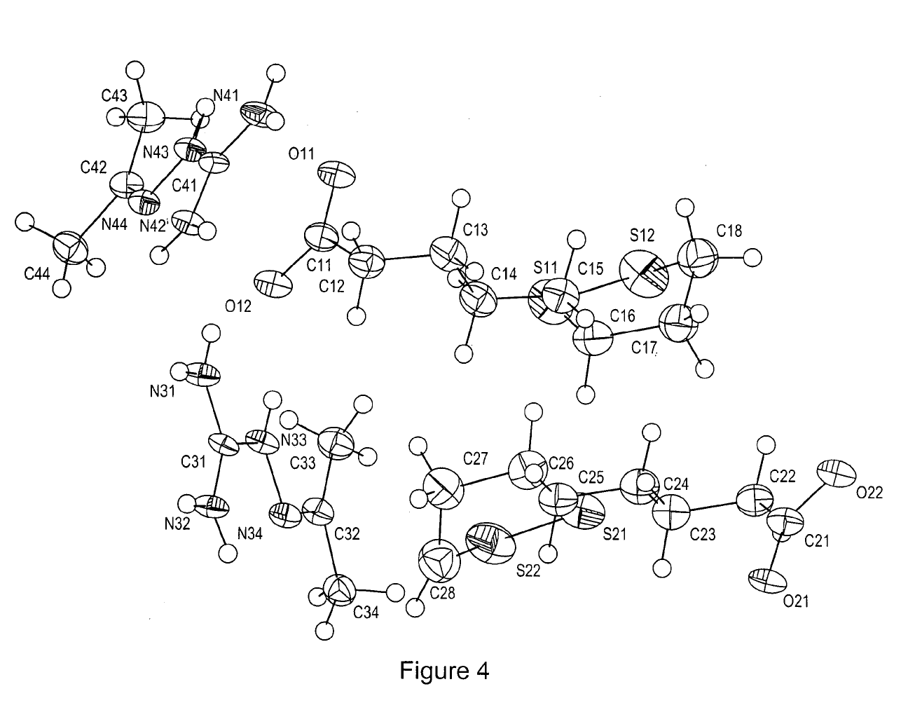

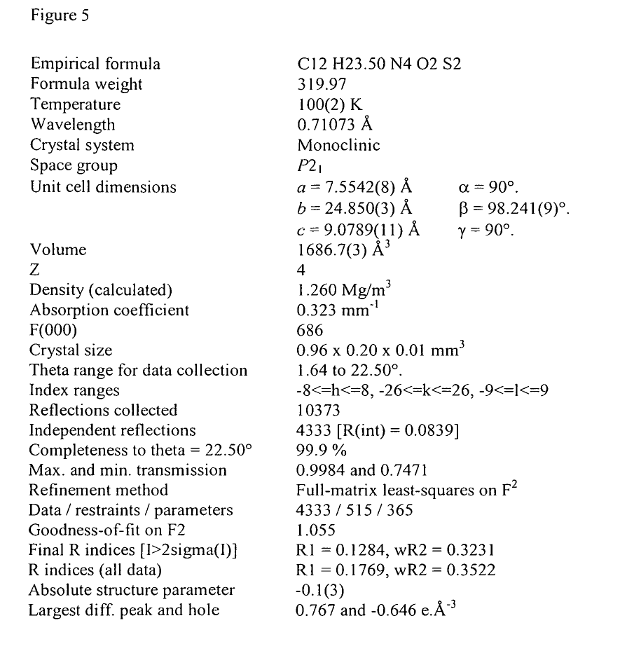

The crystal structure of the re-crystallized salt of Example 2 was determined using standard methods. The crystal structure is shown in FIG. 4. The structural characteristics are enumerated in the table of FIG. 5.

### Example 4

### Power XRD Analysis of the Lipoic Acid Salt of the Compound of Formula III

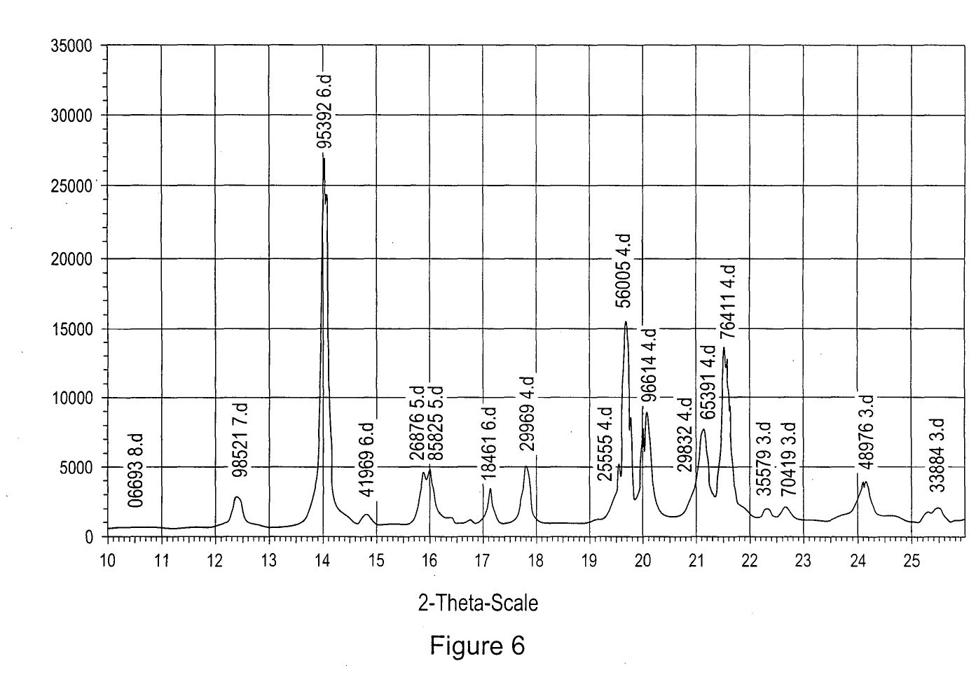

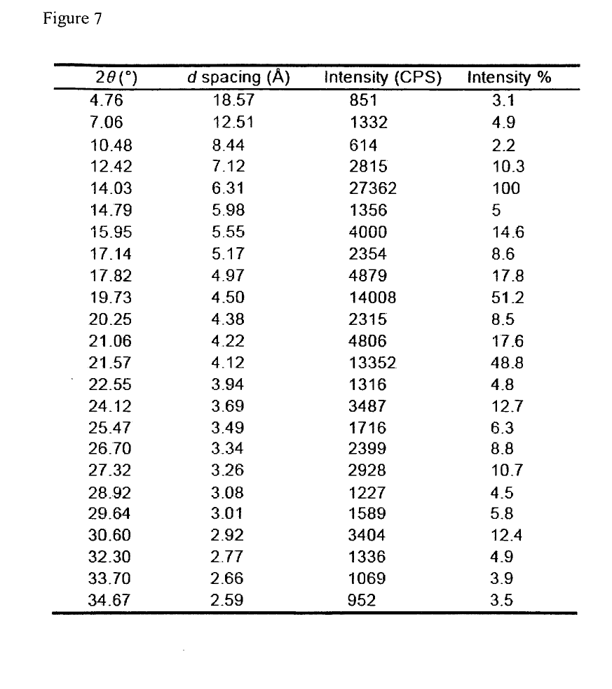

The Power XRD (X-ray diffraction) pattern of the recrystallized salt of Example 2 was determined using standard methods, and is shown in FIG. 6. The peak assignments and the absolute and relative intensities in the powder XRD are shown in FIG. 7.

### Example 5

### Differential Scanning Calorimetry (DSC) and Thermogravimetric Analysis (TGA) of the Lipoic Acid Salt of the Compound of Formula III

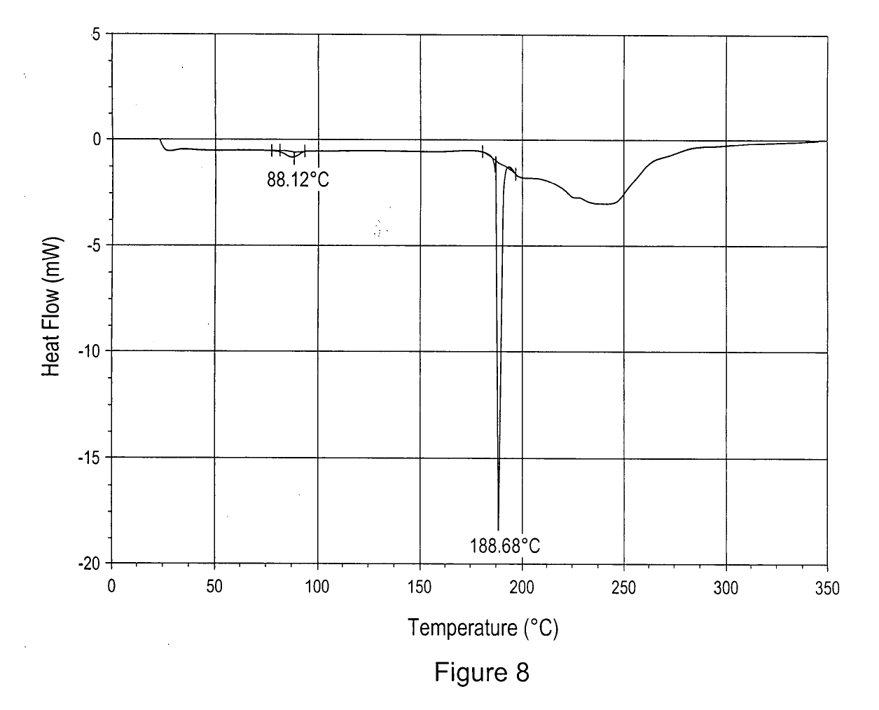

FIG. 8 depicts Differential Scanning Calorimetry (DSC) of the recrystallized salt of Example 2. From the DSC thermogram, it can be seen that the crystals undergo an endothermic phase transition at 88° C. In addition, the crystals show a sharp melting point at 188.7° C. The compound decomposes soon after melting. The decomposition endotherm is broad and spans the temperature range 190-290° C.

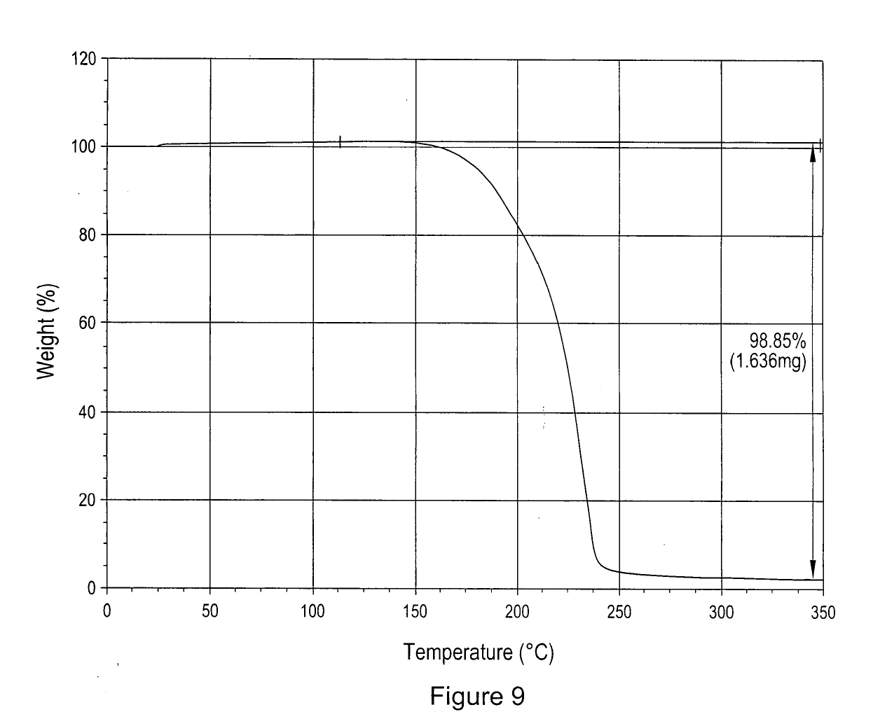

FIG. 9 is a graph showing the Thermogravimetric Analysis (TGA) analysis of the recrystallized salt of Example 2. The TGA analysis indicates that, in the open pan, the complete decomposition of the compound begins at 150° C. and ends at 250° C. Furthermore, no other transitions were associated with the compound. From this one may infer that there is no solvent loss at all. Finally, it was observed that the compound totally decomposes by the end of the run, within experimental error.

### Example 6

### In vitro Pharmacology Analysis of the Lipoic Acid Salt of the Compound of Formula III

The IC₅₀ of the lipoic acid salt of the compound of Formula III was determined for each of three assays.

First, an inducible NOS activity assay was performed on the recrystallized salt of Example 2. This assay measures the formation of nitrite from arginine using an enzyme isolated from LPS-+INFγ-treated mouse macrophages. In this assay, the test salt (as a 10-fold concentrated solution in H₂O), reference compound or water (control) are incubated for 180 min at 37° C. with the enzyme (0.5 U) in a buffer containing 40 mM Tris-HCl (pH 8.0), 0.5 mM NADPH, 4 μM FAD, 12 μM BH₄, 3 mM DTT and 0.1 mM L-arginine. For basal control measurements, the enzyme is omitted from the incubation medium. Following incubation, Griess reagent containing 0.05% naphtylene diamine, 0.5% sulfanilamide and 2.5% orthophosphoric acid is added and the samples are incubated for 10 min at 22° C. The amount of nitrite produced is then quantified with a microplate reader (Spectrafluorplus, Tecan) by measuring the absorbance at λ=550 nm. The results are expressed as IC50 in M. This assay may be performed using the standard inhibitory reference compound, I400 W, which may be tested in each experiment at several concentrations to obtain an inhibition curve from which its IC₅₀ value is calculated. Further information regarding this protocol may be found in Tayeh and Marletta (1989), Macrophage oxidation of L-arginine to nitric oxide, nitrite, and nitrate, J. Biol. Chem., 264: 19654. In this assay, the salt displayed an IC₅₀ of 3.7E-05 M and an n_{H} of 0.9.

In addition, the effect on superoxide O2 secretion was measured. This assay quantifies the secretion of superoxide O₂⁻ from phorbol 12-mysirate 13-acetate (PMA)-stimulated human HL-60 cells, by the measurement of cytochrome C reduction. The test salt (as a 10-fold concentrated solution in H₂O), reference compound or water (control) are pre-incubated for 15 min at 37° C. with HL-60 cells (5×10⁵ cells) suspended in a buffer containing 137 mM NaCl, 2.68 mM KCl, 0.9 mM CaCl₂, 0.5 mM MgCl₂, 8.1 mM Na₂HPO₄, 1.47 mM KH₂PO₄ (pH 7.4 ) and 19 μM cytochrome C. The absorbance is then measured at λ=550 nm using a spectrophotometer to detect any compound interference with the photometric detection at this wavelength. Thereafter, the reaction is initiated by the addition of 30 nM PMA and the mixture is incubated for 15 min at 37° C. in the dark. For basal control measurements, the incubation medium also contains 275 U/ml superoxyde dismutase (SOD) to catalyze the destruction of superoxide O₂⁻. Following incubation, the mixture is cooled to 4° C., centrifuged at 250 g for 5 min and the supernatants are collected. The absorbance is then measured at λ=550 nm and the activity is determined by subtracting signal measured in the presence of SOD from that measured in its absence. This assay may be performed using the standard inhibitory reference compound, diphenyleneiodonium, which may be tested in each experiment at several concentrations to obtain an inhibition curve from which its IC₅₀ value is calculated. Further information about the protocol may be found in Lorico et al. (1986), Gentisic acid: an aspirin metabolite with multiple effects on human blood polymorphonuclear leukocytes, Biochem. Pharmacol., 35 : 2443. In this assay, the salt displayed an IC₅₀ of 3.1E-04 M.

Furthermore, the effect on lipid peroxidation quantified by the measurement of ascorbic acid-induced production of malonaldehyde in rat liver microsomes. Specifically, homogenates of liver microsomes (150 μg) are pre-incubated for 5 min at 37° C. with the test salt (as a 10-fold concentrated solution in H₂O), reference compound or water (control) in a buffer containing 300 mM NaCl, 0.1 mM FeCl₃ and 8 mM NaH₂PO₄/Na₂HPO₄ (pH 7.4). Thereafter, the reaction is initiated by the addition of 0.1 mM ascorbic acid and the mixture is incubated for 20 min at 37° C. For basal control measurements, ascorbic acid is omitted from the incubation medium. These measurements are also used to detect any compound interference with the photometric detection at the selected wavelength. Following incubation, the reaction is stopped by the addition of 5 mM EDTA/NaOH. Lipid peroxides are extracted by the addition of 1% 2-thiobarbituric acid and 2.8% trichloroacetic acid followed by heating to 100° C. for 15 min then cooling to 4° C., addition of n-butanol-1 and centrifugation at 1200× g for 5 min. The amount of lipid peroxides present in the supernatant is quantified by measuring the absorbance at λ=532 nm using a spectrophotometer. This assay may be performed using the standard inhibitory reference compound N-propyl gallate, which may be tested in each experiment at several concentrations to obtain an inhibition curve from which its IC₅₀ value is calculated. Additional details on this assay may be found in Aruoma et al. (1990), An evaluation of the antioxidant and potential pro-oxidant properties of food additives and of trolox C, vitamin E and probucol, Free Rad. Res. Commun., 10 : 143. In this assay, the salt displayed an IC₅₀ of 1.8E-03 M.

### EQUIVALENTS

The present disclosure provides among other things compositions and methods for treating NOS-associated diseases. While specific embodiments of the subject disclosure have been discussed, the above specification is illustrative and not restrictive. Many variations of the systems and methods herein will become apparent to those skilled in the art upon review of this specification. The full scope of the claimed systems and methods should be determined by reference to the claims, along with their full scope of equivalents, and the specification, along with such variations.

### INCORPORATION BY REFERENCE

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

1. A lipoic acid salt of a compound of Formula I: 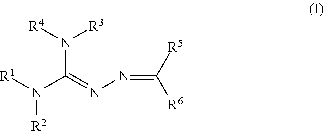
having a lipoate ion enriched for the R-(+) enantiomer, and wherein
R¹ and R², each independently, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, and a macromolecule, or R¹ and R², taken together, form ═C═O, ═CH—CHO, or ═C(R⁷)(R⁸);
R³ and R⁴, each independently, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, and a macromolecule, or R³ and R⁴, taken together, form ═C═O, ═CH—CHO, or ═C(R⁷)(R⁸);
R⁵ and R⁶, each independently, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, thioether, and a macromolecule;
R⁷ and R⁸, each independently, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, thioether, and a macromolecule.

2. The lipoic acid salt of claim 1, wherein R¹ is H, R² is H, R³ is H, R⁴ is H, and
R⁵ and R⁶, each independently, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, thioether, thioketone, and a macromolecule.

3. The lipoic acid salt of claim 2, wherein the lower alkyl is a C₁-C₆-alkyl.

4. The lipoic acid salt of claim 1, wherein the acyl is a formyl.

5. The lipoic acid salt of claim 1, wherein the macromolecule is a polypeptide or oligopeptide.

6. The lipoic acid salt of claim 5, wherein the polypeptide is an antibody.

7. The lipoic acid salt of claim 2, wherein R⁵ is CH₃ and R⁶ is CH₃.

8. The lipoic acid salt of claim 1, wherein the salt is in crystalline form.

9. The lipoic acid salt of claim 1, wherein the salt is substantially free of the S enantiomer of lipoate.

10. A pharmaceutical composition comprising a pharmaceutically acceptable carrier and the lipoic acid salt of claim 1.

11-13. (canceled)

14. The pharmaceutical composition of claim 10, which is formulated for systemic administration.

15-17. (canceled)

18. The pharmaceutical composition of claim 10, which is formulated for topical administration.

19. The pharmaceutical composition of claim 10, further comprising at least one of a pharmaceutically acceptable stabilizer, diluent, surfactant, filler, binder, and lubricant.

20. A method of treating a NOS-associated disease comprising, administering to a patient in need thereof a therapeutically effective amount of a lipoic acid salt of a compound of Formula I: 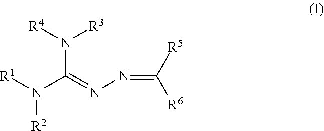
having a lipoate ion enriched for the R-(+) enantiomer, and wherein
R¹ and R², each independently, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, and a macromolecule, or R¹ and R², taken together, form ═C═O, ═CH—CHO, or ═C(R⁷)(R⁸);
R³ and R⁴, each independently, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, and a macromolecule, or R³ and R⁴, taken together, form ═C═O, ═CH—CHO, or ═C(R⁷)(R⁸);
R⁵ and R⁶, each independently, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, thioether, and a macromolecule;
R⁷ and R⁸, each independently, is selected from H, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, thioether, and a macromolecule.

21-22. (canceled)

23. The method of claim 20, wherein the disease is arthritis.

24. The method of claim 20, wherein the disease is Alzheimer's disease, Huntington's disease, or Parkinson's disease.

25. The method of claim 20, wherein the disease is diabetes or a diabetic complication.

26. (canceled)

27. The method of claim 20, wherein the lipoic acid salt is administered systemically.

28. (canceled)

29. A method of treating an NOS-associated disease comprising, administering to a patient in need thereof a therapeutically effective amount of a lipoic acid salt of a compound of Formula II: 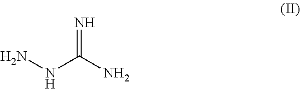
having a lipoate ion enriched for the R-(+) enantiomer.

30. The method of claim 29, wherein the lipoic acid salt is substantially free of the S enantiomer of lipoate.

31-39. (canceled)
